(19) United States
(12) Patent Application Publication
(10) Pub. No.: US 2012/0213714 A1
(43) Pub. Date: Aug. 23, 2012

(54) Method and compositions as an adjunct for the prevention and treatment of periodontal disease

(75) Inventors: Chifu Huang, Lexington, KY (US); Jeffrey Lee Ebersole, Lexington, KY (US)

(73) Assignee: Chifu Huang, Lexington, KY (US)

(21) Appl. No.: 13/033,585

(22) Filed: Feb. 23, 2011

(51) Int. Cl. A61K 8/92; A61Q 11/00

(52) U.S. Cl. 424/49

(57) ABSTRACT

The present invention discloses compositions of omega fatty acids as an adjunct for controlling and preventing periodontal disease. The application of these compositions can be used for the preparation of drugs, dietary supplements, food, nutrition, and daily necessities with oral health care and therapeutic effects.

### FIELD OF THE INVENTION

The present invention relates to compositions of omega-3 fatty acids, eicosapentanoic acid (EPA, C-20) and α-linolenic acid (ALA, C-18), omega-6 fatty acids, γ-linolenic acid (GLA, C18), and other active fatty acids, capric acid (C10), lauric acid (C12), myristic acid (C14), with antimicrobial activity against the bacteria implicated in periodontal infections and disease. The compositions of the fatty acids are used as an adjunct for the prevention and treatment of caries disease.

### BACKGROUND OF THE INVENTION

Fish oil, often used synonymously with the components of omega-3 polyunsaturated fatty acids (PUFA), is widely used as dietary or nutritional supplement. Biologically active omega-3 fatty acids mainly include eicosapentaenoic acid (EPA), docosahexaenoic acid (DHA), α-linolenic acid (ALA). Their common feature, the first double bond in the carbon chain located at the third position, has played an important role in a range of physiological functions. Other omega-6 fatty acids, such as arachidonic acid (ARA) and γ-linolenic acid (GLA), and omega-9 fatty acid also play important physiological functions in humans.

For example, omega-3 fatty acids, eicosapentaenoic acid (EPA) and docosahexaenoic acid (DHA), have been suggested to contribute varied health benefits, primarily related to their ability to minimize inflammation, and actions as antioxidants. It has also been reported that EPA and DHA can serve as precursors to eicosanoids end products, which can resolve inflammation. Recent studies have shown that n-3 PUFA can reduce periodontal inflammation and bone resorption in the oral cavity of rats challenged with specific human oral pathogens. Additionally, studies also reported that fish oil dietary supplementation may have potential benefits as a host modulatory agent in the prevention and/or adjunctive management of periodontitis.

Oral diseases in America have been called a “silent epidemic”. In adults and children, a lack of dental care often results in severe or persistent pain, inability to eat, swollen faces, and increased susceptibility to other medical conditions. The major message of the Surgeon General's report on Oral Health in America is that “oral health is essential to the general health and well-being of all Americans and can be achieved by all Americans.” Dental disease or visits for treatment result in an annual loss of more than 164 million hours of work among adults and more than 51 million school hours among children. Nearly ¼ of adults aged 35 through 44 years have destructive periodontal disease. Periodontal diseases are multifactorial infections elicited by a complex of bacterial species that interact with host tissues and cells causing the release of a broad array of inflammatory cytokines, chemokines, and mediators, some of which lead to destruction of the periodontal structures, including the tooth-supporting tissues, alveolar bone and periodontal ligament. The trigger for the initiation of disease is the presence of complex microbial biofilms that colonize the sulcular regions between the tooth surface and the gingival margin through specific adherence interactions and accumulation due to architectural changes in the sulcus (i.e. attachment loss and pocket formation).

Due to various essential fatty acids found in fish oil, the fatty acids are widely used in functional foods. Our recent discovery reported that omega-3, omega-6, and other fatty acids demonstrating antimicrobial activity against periodontal pathogens. However, no combination of the fatty acids has been specifically used as an adjunct to prevent and treat periodontal disease, potentially missing possible synergistic effect of these agents. There is an urgent need for such oral health products. The present composition, a combination of fatty acids of omega-3, omega-6, and other fatty acids, is applied in products for dental care and used as an adjunctive strategy for managing periodontal infections and disease. The current application can meet the market needs of products specifically targeted to control and prevent periodontitis.

### DISCLOSURE OF INVENTION

The present invention is to provide a novel application and composition of omega fatty acids. The characteristics are the discovery and application of antimicrobial activity of omega-3, omega-6, and other fatty acids against periodontal pathogens. Specifically, the compositions of fatty acids derived from fish oil or algae or plants, have significant antimicrobial activity, allowing them to be applied as adjuncts for the prevention and treatment of periodontitis and gingivitis. The application is a novel use of pre-existing compounds which are already proved safe, and there is no prior art of such use.

The invention makes use of the antimicrobial activity of common omega-3 fatty acids (EPA and ALA) and omega-6 fatty acid (GLA), and other fatty acids, and their application of prevention of oral diseases. The characteristics of the above methods and compounds are:
(1) The inventors discovered that, the omega-3 fatty acids (EPA and ALA), omega-6 fatty acid (GLA), and other fatty acids, capric acid, lauric acid, and myristic acid, have strong antimicrobial bioactivity against oral pathogens. The biological activity has a strong inhibitory effect on oral microorganisms, such as Porphyromonas gingivalis, Candida albicans, Aggregatibacter actinomycetemcomitans, Fusobacterium nucleatum, but not limited.
(2) Fish oil/omega-3 and omeg-6 fatty acids, can be used as adjunct in the prevention and treatment of periodontal infections. The present invention provides an application for prevention and control of periodontitis by omega-3 and omega-6 fatty acids, and fatty acids, capric acid, lauric acid, myristic acid. The method for prevention and control of oral infections mentioned above is that omega-3 and omega-6 fatty acids, and other fatty acids, ranging from 0.01-20% of the content to the final product weight, where the specific content items are based on the type of system and requirements.

The inventor verified the activity by dissolving 1 mg of omega-3 fatty acids (EPA and ALA) and omega-6 fatty acid (GLA), and fatty acids, capric acid, lauric acid, myristic acid, in 1000 μl ethanol solution; took 1 μl (final concentration 1 μg/ml) and 5 μl (final concentration 5 μg/ml) of solution mixture and added to 100 μl of Porphyromonas gingivalis in medium and cultured overnight at 37° C. After the overnight culture, the medium was diluted 1:10000 times, 5 μl of the diluted culture was applied to blood agar plate and cultured overnight. Calculated the number of colonies on the plates and compared with the control group to obtain the percentage of inhibition. It was found that the 5 μg/ml final concentration of the fatty acid mixture could inhibit 50% of Porphyromonas gingivalis growth. Similarly, the composition of omega-3 fatty acids, omega-6 fatty acids, and the fatty acids can inhibit 50% of Candida albicans at a final concentration of ˜25 μg/ml; inhibit Aggregatibacter actinomycetemcomitans at a final concentration of ˜15 μg/ml.

Oral health care and therapeutic products include medicines, food, and daily necessities. Formulations of these products include the fish oil/omega-3 fatty acids, and also including fish oil fatty acid methyl ester and fish oil fatty acid ethyl ester, their additives. In accordance with these products, they are prepared by conventional production methods.

The composition can be also used in therapeutic formulations such as tablets, sprays, powder, film, paint, ointment, gel, liquid, foam, mouthwash, etc. The treatments mainly refers to the tablets for oral medication in various form of tablets, including oral tablets, bio-adhesive patches, dispersible tablets, effervescent tablets, chewable tablets, tablets and other solutions. The fatty acids, in the above-mentioned application should account for at least 0.1 percent to the weight of the mixture of drugs. The more effective composition would generally be 0.01-20%.

The composition can be also used in functional foods such as chewing gum, beverages, coffee, and other dairy products. The fatty acids should be at least 0.01% to weight mixture of food, although 0.01-20% would be ideal.

The composition can be also used in oral daily necessities such as toothpaste, tooth powder, etc. The fatty acids should be at least 0.01% to the weight of a mixture of daily necessities, although 0.01-20% would be ideal.

### EXAMPLES

The following examples are presented by way of illustration, not of limitation.

### Example 1

Dissolve and mix 1 mg omega-3 or omega-6 fatty acids (EPA, ALA, GLA) and capric acid, lauric acid, myristic acid in 10 ml ethanol to make a 1 mg/ml stock solution. Take 1 μl and 5 μl of above solution mixture and add into 100 μl Porphyromonas gingivalis culture (at final concentration 1 μg/ml and 5 μg/ml), incubate the said bacterial culture overnight at 37° C. Afterwards, dilute the overnight culture by 1:10000. Five μl of the diluted culture mix was applied to blood agar plate and cultured overnight. Calculate the colony numbers on the plate and compared with the negative control plate. Final concentration of 1 μg/ml of omega-3 and omega-6 fatty acids could significantly inhibit the growth of Porphyromonas gingivalis. The result shows that the IC₅₀ of omega-3 fatty acid is approximately 1 μg/ml. IC₅₀ of omega-6 fatty acids (GLA) is similar to 5 μg/ml.

### Example 2

Dissolve 1 mg of the fatty acid composition in 10 ml ethanol. One μl and 5 μl of the above solution were added into 100 μl of Aggregatibacter actinomycetemcomitans culture (at final concentration 1 μg/ml and 5 μg/ml), and cultured overnight at 37° C. Dilute this culture by 1:10000. Five μl of diluted solution was applied to blood agar plate and cultured overnight. Calculate the clone number on the disk, and compared with the negative control. Results showed that the final concentration of 1 μg/ml mixture could inhibit the growth of Aggregatibacter actinomycetemcomitans.

### Example 3

Dissolve 1 mg of fatty acid mixture in 10 ml ethanol. One μl and 5 μl of the mixture were added into 100 μl Candida albicans solution (at final concentration 1 μg/ml and 5 μg/ml), and then cultured overnight at 37° C. Dilute this culture by 1:10000 and apply 5 μl of diluted solution to blood agar plate, and cultured overnight. Calculate the clone number on the plate and compare with the negative control. Results showed that the omega-3 fatty acid methyl esters have significant anti-bacterial activity. Final concentration of 1 μg/ml omega-3 fatty acid methyl ester could inhibit the growth of Candida albicans. The study showed that omega-3 fatty acid methyl esters have IC₅₀ of approximately 2.5 μg/ml.

### Example 4

The weight percentage composition of anti-microbial oral chewing tablet, containing fatty acid mixture, is as follows:

| | |
|---|---|
| Fatty acid mixture | 1% |
| Mannitol/or xylitol or sorbitol | 5% |
| Dextrin | 18.5% |
| Menthol | 0.5% |
| HPMC | 4% |
| Manesium stearate | 0.5% |
| Other ingredients | 80% |

### Example 5

The weight percentage composition of chewing gum, containing fatty acid mixture, is as follows:

| | |
|---|---|
| The fatty acid mixture | 1-5% |
| Gum base | 80% |
| Mannitol, or xylitol or sorbitol | 10% |
| Menthol | 0.2% |
| Lecithin | 0.6% |
| Edible flavors (such as banana or other flavors) | 0.9% |
| Edible citric acid | 0.2% |

### Example 6

The weight percentage composition of chewing gum, containing the fatty acid mixture, is as follows:

| | |
|---|---|
| Fatty acid mixture | 1-5% |
| Gum base | 80% |
| High intense sweetener mogroside, or rebaudisoside | 2% |
| Menthol | 0.2% |
| Lecithin | 0.6% |
| Edible flavors (such as banana or other flavors) | 0.9% |
| Edible citric acid | 0.2% |

### Example 7

The weight percentage composition of dairy products, containing fatty acid mixture, is as follows:

| | |
|---|---|
| Fatty acid mixture | 1-5% |
| Milk | 90-95% |
| Mannitol, or xylitol or sorbitol or syrup | 1-5% |
| Or high intense sweetener | 0.5~1% |

Even though specific examples of the invention are explicitly disclosed herein, the above specifications and specific examples are not restrictive and are illustrative. Upon the review of the specifications with the examples given herein, many variations of the invention will become apparent to those skilled in the art. Therefore, full scope of the invention should be determined by the specifications and by references to the examples along with their full scope equivalents, along with such variations. It is expected that those skilled in the art to employ such variations and the invention to be practiced otherwise than specifically described here. Therefore, the invention includes all modification and variations and equivalents of the subject matter as cited in the attached claims. Henceforth, any combination of the herein described elements in all possible variations is encompassed by the invention.

### BEST MODE FOR CARRYING OUT THE INVENTION

The preferred embodiments of this invention are described herein, which includes the best mode that is known to the inventor for implementing the invention. Even though the invention has been described with reference to particular embodiments, it is to be understood that these embodiments are merely illustrative of the applications and principles of the invention. Therefore, it is to be understood that many modifications of the composition may be made to the illustrative embodiments without departing from the spirit and the scope of the invention as defined in the attached claims.

### INDUSTRIAL APPLICABILITY

The antimicrobial composition of this invention containing of omega polyunsaturated fatty acids is capable of maintaining the original activity against periodontal disease, and can be used in a variety of applications where antimicrobial activity is desired, such as raw materials for consumer goods including oral health products, foods and beverages, functional foods, drugs, pharmaceuticals, dental/medical supplies, or in industrial products. Methods of preparing such compositions, and methods of using such compositions, for example as adjuncts in the treatment and/or prevention of periodontal disease, can be applied in large scale.

1-8. (canceled)

9. A composition for affecting antimicrobial activity against periodontal pathogens, comprising:
at least one fatty acid selected from: an omega-3 fatty acid, an omega-6 fatty acid, a medium-chain fatty acids or their fatty acid esters to a subject,
wherein the fatty acid is formulated in an oral health, medicinal, or food product.

10. A composition of claim 9, wherein the at least one fatty acid/ester is selected from: eicosapentanoic acid (EPA), eicosapentanoic acid methyl ester, eicosapentanoic acid ethyl ester, α-linolenic acid (ALA), α-linolenic acid (ALA) methyl ester, α-linolenic acid (ALA) ethyl ester, γ-linolenic acid (GLA), γ-linolenic acid (GLA) methyl ester, γ-linolenic acid (GLA) ethyl ester, capric acid, lauric acid, and myristic acid.

11. The composition of claim 10, wherein the at least one fatty acid/ester is eicosapentanoic acid (EPA), or eicosapentanoic acid (EPA) methyl ester, or eicosapentanoic acid (EPA) ethyl ester.

12. The composition of claim 11, wherein the oral health, medicinal, or food product is selected from a chewable/chewing gum, a chewable/chewing tablets, a toothpaste, a mouthwash, a spray, a powder, a film, a paint, an ointment, a gel, a liquid, a foam, a paste, a tablet, a gum, a solution, a chew, a tooth powder, a beverage, a candy, and a dairy product.

13. The composition of claim 11, and further comprising a second fatty acid selected from α-linolenic acid (ALA), α-linolenic acid (ALA) methyl ester, α-linolenic acid (ALA) ethyl ester, γ-linolenic acid (GLA), γ-linolenic acid (GLA) methyl ester, γ-linolenic acid (GLA) ethyl ester, capric acid, lauric acid, and myristic acid.

13. The composition of claim 11, and further comprising at least one additional component, selected from: vitamin A, vitamin C, vitamin D, vitamin E, Lo Han Kuo extract, stevia, and xylitol.

14. The composition of claim 9, wherein the oral health, medicinal, or food product is selected from a chewable/chewing gum, a chewable/chewing tablets, a toothpaste, a mouthwash, a spray, a powder, a film, a paint, an ointment, a gel, a liquid, a foam, a paste, a tablet, a gum, a solution, a chew, a tooth powder, a beverage, a candy, and a dairy product.

15. The composition of claim 9, wherein the fatty acids comprise about 0.01% to about 20% weight/weight of the composition.

16. A method of affecting antimicrobial activity against periodontal pathogens in a subject, comprising:
administering a composition of claim 9 to a subject, wherein the subject has or is at risk of developing periodontal disease caused by periodontal pathogens.

17. The method of claim 16, wherein the composition comprises at least one fatty acid selected from: eicosapentanoic acid (EPA), eicosapentanoic acid (EPA) methyl ester, eicosapentanoic acid (EPA) ethyl ester, α-linolenic acid (ALA), α-linolenic acid (ALA) methyl ester, α-linolenic acid (ALA) ethyl ester, γ-linolenic acid (GLA), γ-linolenic acid (GLA) methyl ester, γ-linolenic acid (GLA) ethyl ester, capric acid, lauric acid, and myristic acid.

18. The method of claim 17, wherein the composition comprises eicosapentanoic acid (EPA), or eicosapentanoic acid (EPA) methyl ester, or eicosapentanoic acid (EPA) ethyl ester.

19. The method of claim 16, wherein the subject is identified has having or being at risk of developing periodontal disease caused by periodontal pathogens, for example, Aggregatibacter actinomycetemcomitans, Fusobacterium nucleatum, and Porphyromonas gingivalis, and Candida albicans as well.

20. The method of claim 16, wherein the fatty acids comprise about 0.01% to about 20% weight/weight of the composition.

21. The composition of claim 16, wherein the oral health, medicinal, or food product is selected from a chewable/chewing gum, a chewable/chewing tablets, a toothpaste, a mouthwash, a spray, a powder, a film, a paint, an ointment, a gel, a liquid, a foam, a paste, a tablet, a gum, a solution, a chew, a tooth powder, a beverage, a candy, and a dairy product.

22. A method of affecting antimicrobial activity against periodontal pathogens in a subject, comprising:
administering a composition comprising at least one fatty acid selected from: an omega-3 fatty acid, an omega-6 fatty acid, and a medium-chain fatty acid to a subject, wherein the subject has or is at risk of developing a periodontal disease caused by periodontal pathogens.

23. The method of claim 22, wherein the composition comprises at least one fatty acid selected from: eicosapentanoic acid (EPA), eicosapentanoic acid (EPA) methyl ester, eicosapentanoic acid (EPA) ethyl ester, α-linolenic acid (ALA), α-linolenic acid (ALA) methyl ester, α-linolenic acid (ALA) ethyl ester, γ-linolenic acid (GLA), γ-linolenic acid (GLA) methyl ester, γ-linolenic acid (GLA) ethyl ester, capric acid, lauric acid, and myristic acid.

24. The method of claim 23, wherein the composition comprises eicosapentanoic acid (EPA), or eicosapentanoic acid (EPA) methyl ester, or eicosapentanoic acid (EPA) ethyl ester.

25. The method of claim 24, wherein the composition further comprises a second fatty acid selected from: α-linolenic acid (ALA), α-linolenic acid (ALA), α-linolenic acid (ALA) methyl ester, α-linolenic acid (ALA) ethyl ester, γ-linolenic acid (GLA), γ-linolenic acid (GLA) methyl ester, γ-linolenic acid (GLA) ethyl ester, capric acid, lauric acid, and myristic acid; and/or at least one additional component, selected from: vitamin A, vitamin C, vitamin D, vitamin E, Lo Han Kuo extract, stevia, and xylitol.

26. The method of claim 24, wherein the subject is identified has having or being at risk of developing a by periodontal pathogens, for example, Aggregatibacter actinomycetemcomitans, Fusobacterium nucleatum, and Porphyromonas gingivalis, and Candida albicans as well.

27. The method of claim 22, wherein the composition is formulated in a chewable/chewing gum, a chewable/chewing tablets, a toothpaste, a mouthwash, a spray, a powder, a film, a paint, an ointment, a gel, a liquid, a foam, a paste, a tablet, a gum, a solution, a chew, a tooth powder, a beverage, a candy, and a dairy product.

28. The method of claim 22, wherein the fatty acids comprise about 0.01% to about 20% weight/weight of the composition.
